(19) United States
(12) Patent Application Publication
(10) Pub. No.: US 2008/0139563 A1
(43) Pub. Date: Jun. 12, 2008

(54) OXAZOLIDINONE DERIVATIVES AND METHODS OF USE

(75) Inventors: Roger Tung, Lexington, MA (US); Scott Harbeson, Cambridge, MA (US)

Correspondence Address: EDWARDS ANGELL PALMER & DODGE LLP, P.O. BOX 55874, BOSTON, MA, 02205, US

(73) Assignee: Concert Pharmaceuticals Inc., Lexington, MA (US)

(21) Appl. No.: 11/877,666

(22) Filed: Oct. 23, 2007

Related U.S. Application Data
(60) Provisional application No. 60/853,890, filed on Oct. 23, 2006.
(60) Provisional application No. 60/974,637, filed on Sep. 24, 2007.

(51) Int. Cl. A61K 31/5355; C07D 413/10; A61P 31/04

(52) U.S. Cl. 514/236.8; 544/139

(57) ABSTRACT

This invention relates to novel N-[[3-[3-Fluoro-4-(4-morpholinyl)phenyl]-2-oxo-5-oxazolidinyl]methyl]-acetamide derivatives, their acceptable acid addition salts, solvates and hydrates. The invention also provides compositions comprising a compound of this invention and the use of such compositions in methods of treating diseases and conditions beneficially treated by antimicrobial agents.

### RELATED APPLICATIONS

This application claims the benefit of U.S. provisional patent application Nos. 60/853,890, filed Oct. 23, 2006, and 60/974,637, filed Sep. 24, 2007. The contents of these applications are incorporated herein by reference in their entirety.

### TECHNICAL FIELD OF THE INVENTION

This invention relates to novel N-[[3-[3-Fluoro-4-(4-morpholinyl)phenyl]-2-oxo-5-oxazolidinyl]methyl]-acetamide derivatives, their acceptable acid addition salts, solvates, and hydrates and thereof. The invention also provides compositions comprising a compound of this invention and the use of such compositions in methods of treating diseases and conditions beneficially treated by antimicrobial agents.

### BACKGROUND OF THE INVENTION

Linezolid is the generic name for (S)—N-[[3-[3-Fluoro-4-(4-morpholinyl)phenyl]-2-oxo-5-oxazolidinyl]methyl]-acetamide. It has been shown to be effective in a number of animal models as an anti-microbial agent. The PK/PD relationship established in a mouse thigh infection model showed that the major parameter determining efficacy was the time above MIC. Linezolid is known to be a useful antimicrobial agent that is effective against a number of human and veterinary pathogens, including Gram-positive bacteria and certain Gram-negative and anaerobic bacteria. See U.S. Pat. No. 5,688,792 and International Application No. WO 95/07271.

In clinical trials, linezolid has been shown effective in the treatment of the following infections: Vancomycin-Resistant Enterococcus faecium; Nosocomial pneumonia due to Staphylococcus aureus and Streptococcus pneumoniae; complicated skin and skin structure infections caused by Staphylococcus aureus, Streptococcus pyogenes, or Streptococcus agalactiae; uncomplicated skin and skin structure infections caused by Staphylococcus aureus or Streptococcus pyogenes; and community-acquired pneumonia caused by Streptococcus pneumoniae or Staphylococcus aureus. (Barbachyn, M R et al., U.S. Pat. No. 5,688,792 to Pharmacia & Upjohn Co.; ZYVOX Label revised July 2006).

The recommended human dose is 600 mg every 12 hours for Vancomycin-resistant Enterococcus faecium, including bacteremia; nosocomial pneumonia; complicated skin and skin structure infections; and community-acquired pneumonia, including bacteremia. A dose of 400 mg BID is recommended for uncomplicated skin and skin structure infections. In clinical trials, this dose was shown to exceed the MIC90 for Staphylococcus aureus at trough. The PK/PD relationship in humans has not been clearly established. In one study, AUC/MIC was found to be the efficacy predictor; however, this PK/PD predictor was considered to be not reliable. Linezolid shows nonlinear kinetics at higher doses. Doses of 725 mg three times a day could not be tolerated due to an increase in serum creatinine. Myelosuppression has been reported in patients receiving linezolid. The myelosuppression is reversible and patients receiving linezolid should be monitored weekly. Although the PK/PD relationship for linezolid in humans is not well established, it would clearly be advantageous to identify a compound with a longer serum half-life that could maintain exposure levels above MIC at similar or lower doses. This would allow for a lower BID dose while maintaining the required MIC or for administration of higher dosage QD which would maintain the required MIC, while reducing AUC.

Metabolism of linezolid has been studied in mice, rats, dogs and humans where two major metabolic pathways have been identified. The major metabolites excreted are the carboxylic acids known as M4 and M6 resulting from hydrolysis of the lactone and lactam rings, respectively, that are formed by oxidations of the morpholine group. These metabolites are inactive. In humans, the principal metabolic pathway is the lactone pathway. See Slatter, J G et al., Xenobiotica 2002, 32, p. 907 and Drug Metab Dispos 2001, 29, p. 1136. Approximately 35% of an administered dose in humans is found in the urine as the parent compound while 50% of the dose is accounted for as the two metabolites. The oxidation of the morpholine ring is not due to Cyp enzymes. In vitro studies showed that linezolid is not a substrate, inhibitor, or inducer of clinically relevant Cyp isoforms (1A2; 2C9; 2C19; 2D6; 2E1; 3A4). See US NDA No. 02130.

The N-oxide of linezolid is also being investigated in pre-clinical trials as an anti-bacterial agent.

It is therefore desirable to create a compound displaying the beneficial activities of linezolid, that may also have other benefits, e.g., reduced adverse side effects, with a decreased metabolic liability, to further extend its pharmacological effective life, enhance patient compliance and, potentially, to decrease population pharmacokinetic variability and/or decrease its potential for dangerous drug-drug interactions.

### BRIEF DESCRIPTION OF THE DRAWINGS

FIG. 1 depicts the serum pharmacokinetics of a combination of linezolid and Compound 100 following intravenous infusion into a female chimpanzee.

FIG. 2 depicts the serum pharmacokinetics of a combination of linezolid and Compound 100 following intravenous infusion into a male chimpanzee.

FIG. 3 depicts the serum pharmacokinetics of a combination of linezolid and Compound 100 following oral administration to a female chimpanzee.

FIG. 4 depicts the serum pharmacokinetics of a combination of linezolid and Compound 100 following oral administration to a male chimpanzee.

### DEFINITIONS

The terms “ameliorate” and “treat” are used interchangeably and both mean decrease, suppress, attenuate, diminish, arrest, or stabilize the development or progression of a disease (e.g., an infection, microbe).

By “disease” is meant any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of linezolid will inherently contain small amounts of deuterated and/or ¹³C-containing isotopologues. The concentration of naturally abundant stable hydrogen and carbon isotopes, notwithstanding this variation, is small and immaterial with respect to the degree of stable isotopic substitution of compounds of this invention. See for instance Wada E and Hanba Y, Seikagaku 1994 66: 15; Ganes L Z et al., Comp. Biochem. Physiol. A Mol. Integr. Physiol. 1998 119: 725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation) at each atom designated as deuterium in said compound.

The term “isotopic enrichment factor” as used herein means the ratio between the isotopic abundance and the natural abundance of a specified isotope.

In other embodiments, a compound of this invention has an isotopic enrichment factor for each atom designated as deuterium in Formula I or Ia of at least 3500 (52.5% deuterium incorporation at each atom designated as deuterium), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition.

In another embodiment, a “compound”, as defined herein, contains less than 10%, preferably less than 6%, and more preferably less than 3% of all other isotopologues combined, including a form that lacks any deuterium or ¹³C. In certain aspects, the compound contains less than “X”% of all other isotopologues combined, including a form that lacks any deuterium or ¹³C; where X is any number between 0 and 10 (e.g., 1, 0.5, 0.001), inclusive. Compositions of matter that contain greater than 10% of all other isotopologues combined are referred to herein as “mixtures” and must meet the parameters set forth below. These limits of isotopic composition and all references to isotopic composition herein refer solely to the relative amounts of deuterium/hydrogen and ¹³C/¹²C present in the active, free base form of the compound of Formula I/Ia, and do not include the isotopic composition of hydrolyzable portions of counterions.

The term “isotopologue” refers to species that differ from a specific compound of this invention only in the isotopic composition of their molecules or ions.

The term “compound” as used herein, is also intended to include salts, solvates, or hydrates, thereof. The specific recitation of “salt,” “solvate,” or “hydrate,” in certain aspects of the invention described in this application shall not be interpreted as an intended omission of these forms in other aspects of the invention where the term “compound” is used without recitation of these other forms.

A salt of a compound of this invention is formed between an acid and a basic group of the compound, such as an amino functional group, or a base and an acidic group of the compound, such as a carboxyl functional group. According to another preferred embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric, hydrobromic, hydroiodic, sulfuric and phosphoric acid, as well as organic acids such as para-toluenesulfonic, salicylic, tartaric, bitartaric, ascorbic, maleic, besylic, fumaric, gluconic, glucuronic, formic, glutamic, methanesulfonic, ethanesulfonic, benzenesulfonic, lactic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylenesulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces.

The compounds of the present invention may contain one or more asymmetric carbon atoms. As such, a compound of this invention can exist as the individual stereoisomers (enantiomers or diastereomers) as well a mixture of stereoisomers. Accordingly, a compound of the present invention will include not only a stereoisomeric mixture, but also individual respective stereoisomers substantially free of other stereoisomers. The phrase “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present Methods of obtaining or synthesizing diastereomers are well known in the art and may be applied as practicable to final compounds or to starting material or intermediates. Other embodiments are those wherein the compound is an isolated compound. The term “at least X % enantiomerically enriched” as used herein means that at least X % of the compound is a single enantiomeric form, wherein X is a number between 0 and 100, inclusive.

The term “stable compounds”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to atypical antipsychotic agents).

Both “²H” and “D” refer to deuterium. “Stereoisomer” refers to both enantiomers and diastereomers. “tert” refers to tertiary. “CDI” refers to 1,1′-carbonyldiimidazole.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Throughout this specification, reference to “each Y” includes, independently, all “Y” groups (e.g., Y¹, Y², Y³, and Y⁴) where applicable; “each W” includes, independently, all “W” groups (e.g., W¹, W², W³, W⁴, and W⁵) where applicable; “each Z” includes, independently, all “Z” groups (e.g., Z¹, Z², Z³, and Z⁴) where applicable.

### Therapeutic Compounds

The present invention provides a compound of formula I or Ia:

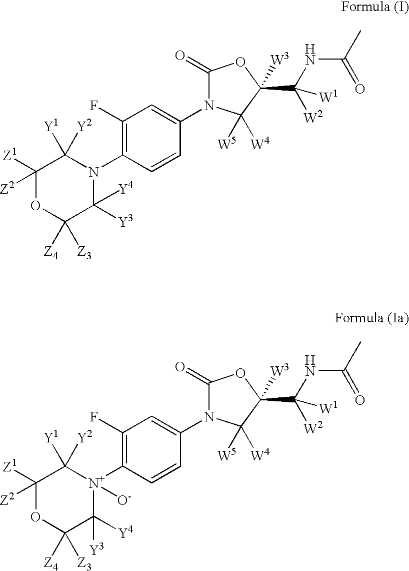

or a salt of Formula I; or a hydrate or solvate of Formula I or Ia, wherein:

each W is independently hydrogen or deuterium;

each Y is independently hydrogen or deuterium;

each Z is independently hydrogen, deuterium, or fluorine; and

at least one W, Y or Z is deuterium.

In one embodiment at least one W is deuterium; at least two Y moieties are deuterium; and at least two Z moieties are deuterium or fluorine.

In one embodiment, W¹ and W² are simultaneously deuterium.

In one embodiment, Y¹, Y², Y³ and Y⁴ are simultaneously deuterium.

In one embodiment, each of Z¹, Z², Z³ and Z⁴ is independently selected from deuterium and fluorine. In a more specific embodiment, Z¹, Z², Z³ and Z⁴ are simultaneously deuterium.

In certain embodiments, the configuration of the compound of Formula I or Ia is (S).

In a more specific embodiment, Y¹, Y², Y³, Y⁴, W¹ and W² are simultaneously deuterium.

In another specific embodiment, each of Z¹, Z², Z³ and Z⁴ is independently selected from deuterium and fluorine; and W¹ and W² are simultaneously deuterium.

In another specific embodiment, Y¹, Y², Y³, Y⁴, Z¹, Z², Z³ and Z⁴ are simultaneously deuterium. In another specific embodiment, Y¹, Y², Y³, Y⁴, Z¹, Z², Z³ and Z⁴ are simultaneously deuterium; and W³, W⁴ and W⁵ are simultaneously hydrogen.

In still another specific embodiment, Z¹, Z², Z³ and Z⁴ are simultaneously fluorine; and Y¹, Y², Y³ and Y⁴ are simultaneously deuterium.

In yet another specific embodiment Y¹, Y², Y³, Y⁴, Z¹, Z², Z³, Z⁴, W¹ and W² are simultaneously deuterium.

In another embodiment, Z¹, Z², Z³ and Z⁴ are simultaneously fluorine; and Y¹, Y², Y³, Y⁴, W¹, and W² are simultaneously deuterium.

In another specific embodiment, Z¹, Z², Z³ and Z⁴ are simultaneously deuterium; and Y¹, Y², Y³, Y⁴, W¹ and W² are simultaneously hydrogen. In another specific embodiment, Z¹, Z², Z³ and Z⁴ are simultaneously deuterium; and W³, W⁴ and W⁵ are simultaneously hydrogen.

In still another specific embodiment, Z¹, Z², Z³, Z⁴, Y¹, Y², Y³ and Y⁴ are simultaneously deuterium; and W¹ and W² are simultaneously hydrogen.

In one embodiment, the compound of formula I or Ia contains at least three deuterium atoms.

In one embodiment, the compound of formula I or Ia contains at least four deuterium atoms.

In one embodiment, the compound of formula I or Ia contains at least five deuterium atoms.

Examples of specific compounds of this invention include:

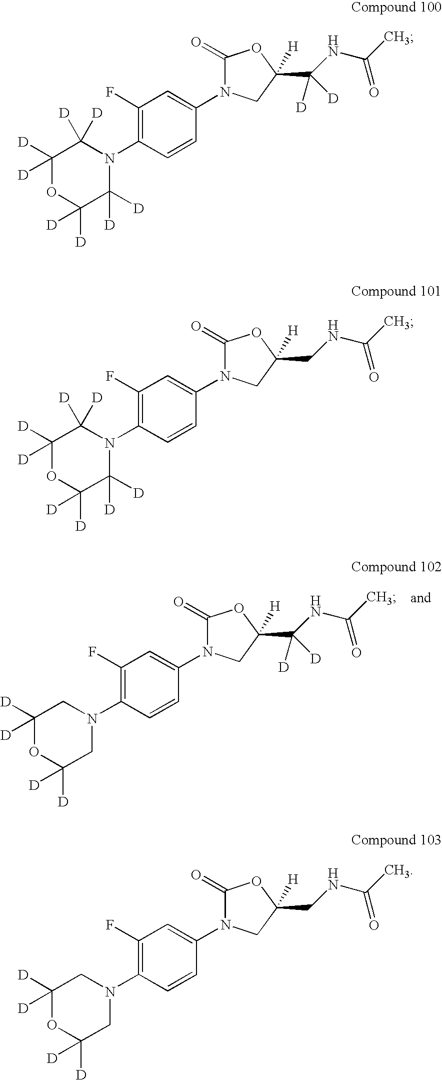

Other examples of specific compounds of this invention include the following:

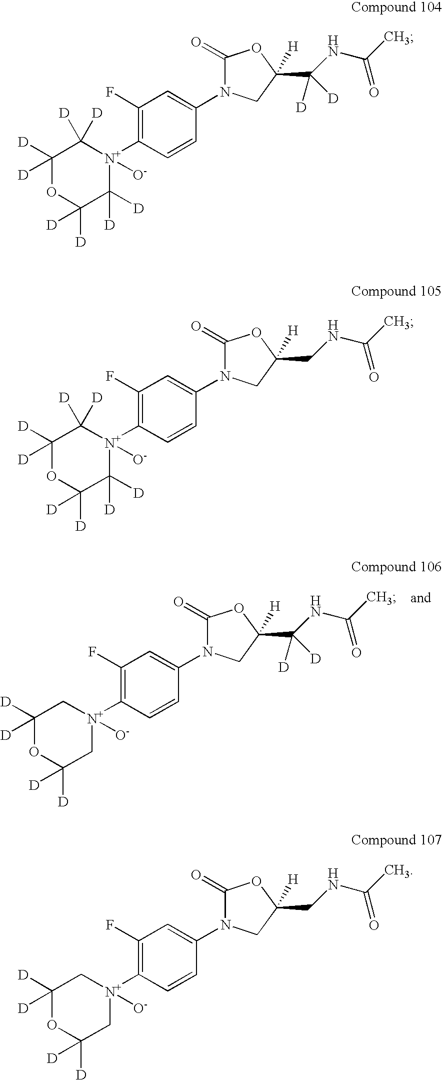

In another set of embodiments, any atom not designated as deuterium in any of the embodiments set forth above is present at its natural isotopic abundance.

General methods of incorporating deuterium in similar compounds are extensively documented. See, for instance, The Journal of Labelled Compounds and Radiopharmaceuticals (John Wiley & Sons), most issues of which contain detailed experimental descriptions on specific incorporation of deuterium into bioactive small organic molecules. See also, for instance, Leis H J Curr. Org. Chem. 1998 2: 131 and reference therein, and Moebius G, ZfI-Mitteilungen 1989 150: 297. Suitable commercial supplies of deuterium-labeled reagents include, among others, Isotec, Inc. (Miamisburg, Ohio); Cambridge Isotope Laboratories (Andover, Mass.); ICON Services Inc. (Summit, N.J.); and C/D/N Isotopes, Inc. (Pointe-Claire, Quebec, Canada).

The synthesis of compounds of formula I/Ia can be readily effected by synthetic chemists of ordinary skill by means known in the art of organic synthesis. Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Relevant procedures and intermediates are disclosed, for instance in PCT publications WO1997010223, WO2005099353, WO1995007271, WO2006008754; Lizondo, J et al., Drugs Fut 1996, 21(11):1116; Brickner, S J et al., J Med Chem 1996, 39(3):673; and Mallesham, B et al., Org Lett 2003, 5(7):963. The scheme below illustrates how compounds of formula I or Ia may be prepared.

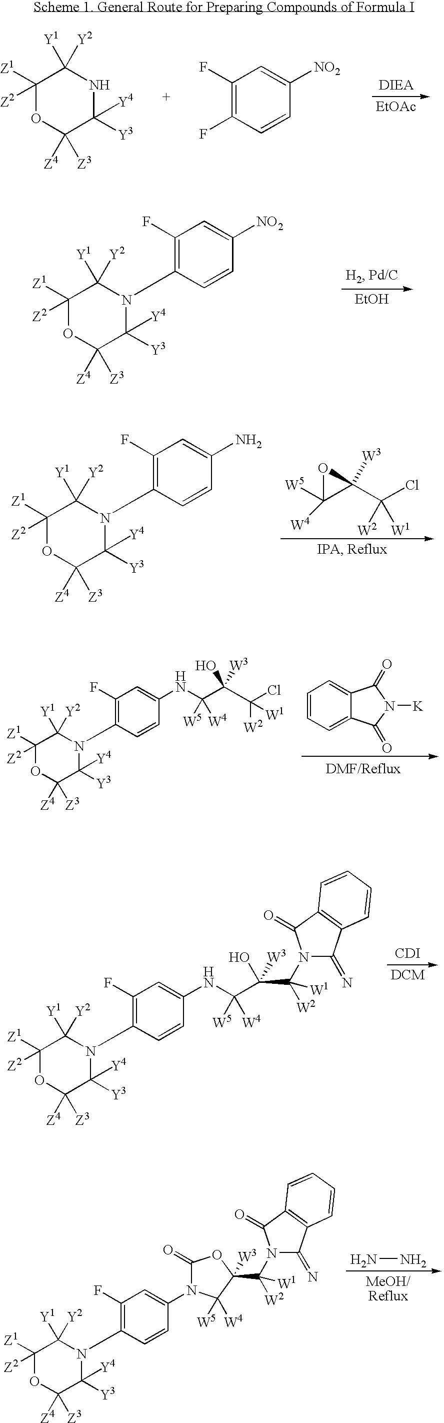

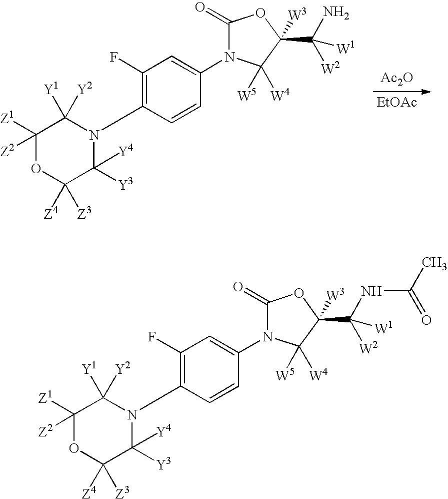

Scheme 1 above shows a general route for preparing compounds of formula I. Compounds of formula Ia can be made from Formula I compounds using a suitable oxidant such as pertrifluoroacetic acid or m-chloroperbenzoic acid. See WO 1997010223. Other approaches to synthesizing compounds of formula I/Ia are set forth in the examples or can readily be adapted from references cited herein. Variations of these procedures and their optimization are within the skill of the ordinary practitioner

The specific approaches and compounds shown above are not intended to be limiting. Additional methods of synthesizing compounds of formula I/Ia and their synthetic precursors, including those within routes not explicitly shown in Schemes herein, are within the means of chemists of ordinary skill in the art. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jahnisch, K et al, Angew. Chem. Int. Ed. Engl. 2004 43: 406; and references therein). In addition to the synthetic references cited herein, reaction schemes and protocols may be determined by the skilled artisan by use of commercially available structure-searchable database software, for instance, SciFinder® (CAS division of the American Chemical Society), STN® (CAS division of the American Chemical Society), CrossFire Beilstein® (Elsevier MDL), or internet search engines such as Google® or keyword databases such as the US Patent and Trademark Office text database. The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3 Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The synthetic methods described herein may also additionally include steps, either before or after any of the steps described in the preceding Scheme, to add or remove suitable protecting groups in order to ultimately allow synthesis of the compound of the formulae described herein. The methods delineated herein contemplate converting compounds of one formula to compounds of another formula. The process of converting refers to one or more chemical transformations, which may be performed in situ, or with isolation of intermediate compounds. The transformations may include reacting the starting compounds or intermediates with additional reagents using techniques and protocols known in the art, including those in the references cited herein. Certain intermediates may be used with or without purification (e.g., filtration, distillation, sublimation, crystallization, trituration, solid phase extraction, and chromatography).

Combinations of substituents and variables envisioned by this invention are only those that result in the formation of stable compounds.

### Compositions

The invention also provides compositions comprising an effective amount of a compound of Formula I/Ia (e.g., including any of the formulae herein), or a pharmaceutically acceptable salt of Formula I; or a hydrate or solvate of Formula I or Ia; and an acceptable carrier. In one embodiment, the composition is pyrogen-free. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Such administration is known to be effective with erectile dysfunction drugs: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of the present invention further comprises a second therapeutic agent. The second therapeutic agent includes any compound or therapeutic agent known to have or that demonstrates advantageous properties when administered with an antimicrobial compound, in particular, in anti-microbial therapy, combination therapy with other anti-microbial and/or anti-inflammatory agents is envisaged. Combination therapies according to the present invention thus include the administration of at least one compound of formula I or Ia, as well as optional use of other anti-microbial agents and optional use of cyclooxygenase inhibitors, particularly selective inhibitors of cyclooxygenase-2. Other anti-microbial therapies and anti-inflammatory agents are described for instance in International Publication No.s WO 01/34128 and WO 03/061704, which applications are incorporated by reference to the extent that they disclose combinations of anti-microbial and anti-inflammatory therapies.

Examples of second therapeutic agents that may be formulated with a compound of this invention include, but are not limited to, gentamicin, tobramycin, aztreonam, cefazolin, ceftazidime, piperacillin, ciprofloxacin, ofloxacin, levofloxacin, celecoxib, and rofecoxib.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

The compounds of the present invention demonstrate a longer half-life, and produce a higher serum concentration level 24 hours post-dosing as compare to the same amount of linezolid on a mole basis. Thus in one embodiment, the invention provides a pharmaceutical composition comprising an effective amount of a compound of formula I, the administration of which to a test subject results in a serum terminal elimination half-life of the compound that is greater than the serum terminal elimination half-life of linezolid when linezolid is administered to an equivalent test subject in a pharmaceutical composition comprising an amount of linezolid that is the same as the amount of the compound of formula I on a mole basis of active ingredient and that is administered in the same dosing regimen as the compound of formula I. In other embodiments, the serum terminal elimination half-life of a compound of formula I is at least 125%, 130%, 135%, 140% or more of the serum terminal elimination half-life of linezolid produced by a corresponding linezolid composition administered in the same dosing regimen.

In a related embodiment, the invention provides a pharmaceutical composition comprising an effective amount of a compound of formula I, or a pharmaceutically acceptable salt thereof, wherein the serum terminal elimination half-life of the compound following administration of a single dose of the first composition to a test subject is greater than 7 hours.

In another embodiment, the invention provides a pharmaceutical composition comprising an effective amount of a compound of formula I, the administration of which to a test subject results in a serum concentration of the compound 24 hours post-administration that is greater than the serum concentration of linezolid 24 hours post-administration when linezolid is administered to an equivalent test subject in a pharmaceutical composition comprising an amount of linezolid that is the same as the amount of the compound of formula I on a mole basis of active ingredient and that is administered in the same dosing regimen as the compound of formula I. In other embodiments, the serum concentration of a compound of formula I produced 24 hours post-administration of a composition of this invention is at least 150%, 175%, 200%, 225%, 250%, 275%, 300% or more of the serum concentration of linezolid produced by a corresponding linezolid composition administered in the same dosing regimen.

In one embodiment, the invention provides a pharmaceutical composition comprising an effective amount of a compound of formula I, the administration of which to a test subject results in an AUC₀₋₂₄ of the compound that is greater than the AUC₀₋₂₄ of linezolid when linezolid is administered to an equivalent test subject in a pharmaceutical composition comprising an amount of linezolid that is the same as the amount of the compound of formula I on a mole basis of active ingredient and that is administered in the same dosing regimen as the compound of formula I. In other embodiments, the AUC₀₋₂₄ produced by a composition of this invention is at least 125%, 130%, 135%, 140%, 145% or more of the AUC₀₋₂₄ produced by a corresponding linezolid composition administered in the same dosing regimen.

The compounds of the present invention also demonstrate greater resistance to certain metabolism as compared to linezolid. Thus, in another embodiment, the invention provides a pharmaceutical composition comprising an effective amount of a compound of formula I, wherein the amount of the compound excreted intact in 24 hours following administration to a test subject is greater than the amount of linezolid excreted intact in 24 hours following administration of linezolid to an equivalent test subject in a pharmaceutical composition comprising an amount of linezolid that is the same as the amount of the compound of formula I on a mole basis of active ingredient and that is administered in the same dosing regimen as the compound of formula I. In other embodiments, the amount of a compound of formula I excreted intact in 24 hours following administration of a composition of this invention is at least 150%, 160%, 170%, 180%, 190%, 200%, 210% or more of the amount of linezolid excreted intact 24 hours following administration of a corresponding linezolid composition administered in the same dosing regimen.

In a related embodiment, the invention provides a pharmaceutical composition comprising an effective amount of a compound of formula I, or a pharmaceutically acceptable salt thereof, wherein in 24 hours following administration of the composition to a subject, at least 45% of the effective amount of the compound is excreted intact by the subject.

In yet another embodiment, the invention provides a pharmaceutical composition comprising an effective amount of a compound of formula I, the administration of which to a test subject results in one or more of: a) a similar AUC₀₋₂₄; b) a similar Cₘₐₓ; or c) a similar Cₘᵢₙ as linezolid when linezolid is administered to an equivalent test subject in a pharmaceutical composition comprising an amount of linezolid that is greater than the amount of the compound of formula I on a mole basis of active ingredient and that is administered in the same dosing regimen as the compound of formula I. In other embodiments, the effective amount of a compound of formula I is less than 80%, 70%, 60%, 50%, 40%, 33%, or less of the amount of linezolid required to produce one or more of a) a similar AUC₀₋₂₄; b) a similar Cₘₐₓ; or c) a similar Cₘᵢₙ when administered in the same dosing regimen as the compound of formula I.

In yet another embodiment, the invention provides a pharmaceutical composition comprising an effective amount of a compound of formula I, the administration of one or more dosages of which to a test subject results in a) maintenance of a serum concentration of the compound at more than 6 mg/L for 24 hours following administration of the first dosage; and b) an AUC₀₋₂₄ of the compound that is less than the AUC₀₋₂₄ of linezolid when linezolid is administered to an equivalent test subject in a pharmaceutical composition comprising an amount of linezolid required to maintain a serum level of linezolid of more than 6 mg/L for 24 hours following administration. In other embodiments, the AUC₀₋₂₄ produced by a composition of this invention is less than 85%, 80%, 75%, 70%, 65%, or less of the AUC₀₋₂₄ produced by the required dosages of the linezolid composition.

In each of the above embodiments, a pharmaceutically acceptable salt of a compound of formula I, and/or linezolid may be used instead of the free base form.

In a more specific embodiment, in each of the compositions set forth above, the compound is selected from compound 100, compound 101, compound 102 or compound 103.

A “test subject” is any mammal, preferably a human.

An “equivalent test subject” is defined herein as being of the same species and sex as the test subject, and which shows no more than 10% variability as compared to the test subject in the pharmacokinetic parameter being tested after administration of an equal amount of linezolid to both the test subject and the equivalent subject.

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537.

Because the compounds of the present invention demonstrate a longer serum half-life than linezolid at equal dosages, they can be administered at lower doses and/or at less frequent intervals than linezolid while still maintaining the required time above minimum inhibitor concentration (“MIC”). As compared with linezolid, less frequent intervals of administration of the compounds of the present invention will reduce the number of spikes in serum concentration that are associated with each dosing. This, in turn, will reduce the patient's total exposure to the compound of this invention over time (cumulative AUC exposure). It is this cumulative AUC exposure that has been implicated in linezolid's progressive toxicity, which is believed to be caused by its inhibition of mitochondrial protein synthesis (Devriese A S et al., Clin Infect Dis 2006, 42:1111). Linezolid's progressive toxicity limits the amount of time that a patient can take the drug.

The reduction in cumulative AUC exposure as compared to linezolid can be further enhanced through the use of controlled release formulations comprising a compound of this invention. Such controlled release formulations are prepared using methods well-known in the art; see e.g. Remington: The Science and Practice of Pharmacy, 21^{st} edition (Lippincott Williams & Wilkins 2005); and Modern Pharmaceutics 4th Edition (Drugs and the Pharmaceutical Sciences Vol. 121), Banker G S and Rhodes C T editors (Informa Healthcare 2002).

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537. An effective amount of a compound of this invention can range from about 50 mg to about 2000 mg every 24 hours, if appropriate in the form of several individual doses. In one embodiment the effective amount of a compound of this invention ranges from about 250 mg to about 1250 mg every 24 hours in the form of a single dosage or two separate dosages of about 125 mg to about 625 mg each given every 12 hours. In another embodiment the effective amount of a compound of this invention ranges from about 750 mg to about 1250 mg every 24 hours in the form of a single dosage or two separate dosages of about 375 mg to about 625 mg each given every 12 hours. In still another embodiment the effective amount of a compound of this invention ranges from about 450 mg to about 1200 mg every 24 hours in the form of a single dosage or two separate dosages of about 225 mg to about 625 mg each given every 12 hours. In a more specific embodiment the effective amount of a compound of this invention ranges from about 450 mg to about 750 mg every 24 hours in the form of a single dosage or two separate dosages of about 225 mg to about 375 mg each given every 12 hours. Other ranges of a compound of this invention that fall within or between any of the above-recited ranges are also within the scope of the invention. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

The milligram amounts of compounds present in the pharmaceutical compositions of the present invention and for use in the methods of the present invention represent the amount of free base compound. It will be understood that the use of pharmaceutical salts of the compounds of the present invention will require that the stated amounts be increased so that a mole equivalent of the free base compound is used.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the second therapeutic agents referenced above will act synergistically with the compounds of this invention. When this occurs, its will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

According to another embodiment, the invention provides a method of treating a subject suffering from or susceptible to a disease that is beneficially treated by linezolid comprising the step of administering to said subject an effective amount of a compound or a composition of this invention. Such diseases are well known in the art and include for instance, the treatment or prevention of a variety of disease states typically treated by antimicrobial therapy (e.g., infection, fungal disorders). The compounds of formula I/Ia, therefore, have utility in the treatment of disorders including those mediated by Gram-positive bacteria and certain Gram-negative and anaerobic bacteria.

In one embodiment, the invention provides a method of treating a subject suffering from or susceptible to an infection caused by a bacteria selected from Enterococcus faecium, Staphylococcus aureus, Streptococcus agalactiae, Streptococcus pneumoniae, Streptococcus pyrogenes, Enterococcus faecalis, Staphylococcus epidermidis, Staphyloccocus haemolyticus, and Pasteurella multocida,

In another embodiment, the invention provides a method of treating a subject suffering from or susceptible to a disease or disorder (or symptoms thereof) selected from a Gram-positive bacterial infection, Vancomycin-resistant Enterococcus faecium infection; nosocomial pneumonia due to Staphylococcus aureus and Streptococcus pneumoniae; complicated skin and skin structure infections caused by Staphylococcus aureus, Streptococcus pyogenes, or Streptococcus agalactiae; uncomplicated skin and skin structure infections caused by Staphylococcus aureus or Streptococcus pyogenes; community-acquired pneumonia caused by Streptococcus pneumoniae or Staphylococcus aureus; and tuberculosis.

In another embodiment, the invention provides a method of treating a subject suffering from or susceptible to a disease or disorder (or symptoms thereof) selected from a Gram-positive bacterial infection, Vancomycin-resistant Enterococcus faecium infection; nosocomial pneumonia due to Staphylococcus aureus and Streptococcus pneumoniae; complicated skin and skin structure infections caused by Staphylococcus aureus, Streptococcus pyogenes, or Streptococcus agalactiae; uncomplicated skin and skin structure infections caused by Staphylococcus aureus or Streptococcus pyogenes; and community-acquired pneumonia caused by Streptococcus pneumoniae or Staphylococcus aureus.

The method of the present invention may also be employed with other therapeutic methods of microbial infection treatment. In particular, in anti-microbial therapy, combination therapy with other anti-microbial and/or anti-inflammatory agents is envisaged. The administration of at least one compound of formula I or Ia as well as optional use of other anti-microbial agents and optional use of cyclooxygenase inhibitors, particularly selective inhibitors of cyclooxygenase-2. Such combination of agents may be administered together or separately and, when administered separately this may occur simultaneously or sequentially in any order, both close and remote in time. Other anti-microbial therapies and anti-inflammatory agents are described for instance in International Publication Nos. WO 01/34128 and WO 03/061704, which applications are incorporated by reference to the extent that they disclose combinations of anti-microbial and anti-inflammatory therapies.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, the invention provides a method of modulating the activity of a cell comprising contacting a cell with one or more compounds of any of the formulae herein.

In another embodiment, the invention provides a method of treating a patient suffering from or susceptible to a bacterial infection comprising the step of administering to the patient in need thereof over a 24 hour period between about 450 mg and about 750 mg of a compound of formula I or Ia. In another embodiment the patient is administered between 450 mg and 700 mg of a compound of formula I or Ia.

In another embodiment, the above method produces a steady state Cₘᵢₙ of greater than about 3 mg/L. In another embodiment, the above method produces a steady state Cₘᵢₙ of greater than about 4 mg/L. In another embodiment, the above method produces a steady state Cₘᵢₙ of greater than about 6 mg/L. In still another embodiment, the above method produces a steady state Cₘₐₓ of less than about 18 mg/L. In another embodiment, the above method produces a steady state Cₘₐₓ of less than about 16 mg/L. In another embodiment, the above method produces a steady state Cₘₐₓ of less than about 13 mg/L. In still another embodiment, the above method produces a steady state Cₘₐₓ of less than about 11.5 mg/L.

In another embodiment, the above method of treatment comprises the further step of co-administering to the patient one or more second therapeutic agents. The choice of second therapeutic agent may be made from any second therapeutic agent known to be useful for co-administration with linezolid.

In a specific embodiment, the combination therapies of this invention include co-administering a compound of Formula I and a second therapeutic agent selected from gentamicin, tobramycin, aztreonam, cefazolin, ceftazidime, piperacillin, ciprofloxacin, ofloxacin, levofloxacin, celecoxib, and rofecoxib.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention comprising both a compound of the invention and a second therapeutic agent to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of formula I or Ia alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of the formulae herein for use in the treatment or prevention in a subject of a disease, disorder or symptom thereof delineated herein.

### Diagnostic Methods and Kits

The compounds and compositions of this invention are also useful as reagents in methods for determining the concentration of linezolid in solution or biological sample such as plasma, examining the metabolism of linezolid and other analytical studies.

According to one embodiment, the invention provides a method of determining the concentration, in a solution or a biological sample, of linezolid, comprising the steps of:
a) adding a known concentration of a compound of Formula I or Ia to the solution of biological sample;
b) subjecting the solution or biological sample to a measuring device that distinguishes linezolid from the compound of Formula I or Ia;
c) calibrating the measuring device to correlate the detected quantity of the compound of Formula I or Ia with the known concentration of the compound of Formula I or Ia added to the biological sample or solution; and
d) measuring the quantity of linezolid in the biological sample with said calibrated measuring device; and
e) determining the concentration of linezolid in the solution of sample using the correlation between detected quantity and concentration obtained for a compound of Formula I or Ia.

Measuring devices that can distinguish linezolid from the corresponding compound of Formula I or Ia include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I or Ia comprising the steps of contacting the compound of Formula I or Ia with a metabolizing enzyme source for a period of time and comparing the amount of the compound of Formula I or Ia with the metabolic products of the compound of Formula I or Ia after the period of time.

In a related embodiment, the invention provides a method of evaluating the metabolic stability of a compound of Formula I or Ia in a patient following administration of the compound of Formula I or Ia. This method comprises the steps of obtaining a serum, urine or feces sample from the patient at a period of time following the administration of the compound of Formula I or Ia to the subject; and comparing the amount of the compound of Formula I or Ia with the metabolic products of the compound of Formula I or Ia in the serum, urine or feces sample.

The present invention also provides kits for use to treat an infectious disease or disorder, including those delineated herein. These kits comprise: a) a pharmaceutical composition comprising a compound of Formula I/Ia or a salt of Formula I; or a hydrate or solvate of Formula I or Ia, wherein said pharmaceutical composition is in a container; and b) instructions describing a method of using the pharmaceutical composition to treat an infectious disease or disorder, including those delineated herein.

The container may be any vessel or other sealed or sealable apparatus that can hold said pharmaceutical composition. Examples include bottles, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. Preferably, the container is a blister pack.

The kit may additionally comprise a memory aid of the type containing information and/or instructions for the physician, pharmacist or subject. Such memory aids include numbers printed on each chamber or division containing a dosage that corresponds with the days of the regimen which the tablets or capsules so specified should be ingested, or days of the week printed on each chamber or division, or a card which contains the same type of information. For single dose dispensers, memory aids further include a mechanical counter which indicates the number of daily doses that have been dispensed and a battery-powered micro-chip memory coupled with a liquid crystal readout and/or audible reminder signal which, for example, reads out the date that the last daily dose has been taken and/or reminds one when the next dose is to be taken. Other memory aids useful in such kits are a calendar printed on a card, as well as other variations that will be readily apparent.

### EXAMPLES

### Example 1

### Synthesis of Intermediate 12

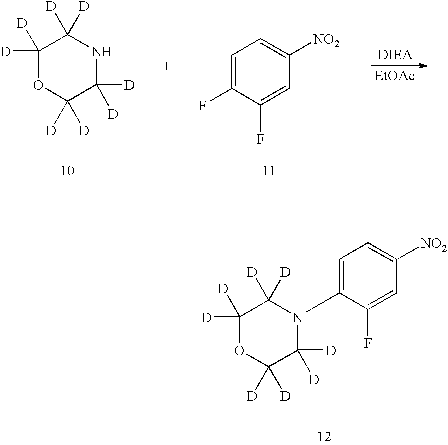

To a stirred solution of d₈-morpholine (10; 23.5 g, 0.25 mol) and ⁱPr₂EtN (44 ml, 0.25 mol) in ethyl acetate (“EtOAc”) (140 ml), cooled in an ice bath, was added 3,4-difluoronitrobenzene (11, 27.4 ml, 0.25 mol), dropwise over 10 min. The reaction mixture was stirred for 48 h at room temperature. The reaction mixture was diluted with EtOAc (300 ml) and CH₂Cl₂ (50 ml) then water (350 ml) was added. The layers were separated and the aqueous layer washed with EtOAc (3×300 ml). The combined organics were dried (MgSO₄), filtered and concentrated in vacuo. The crude product was purified using column chromatography (1 kg silica) eluting with 20% EtOAc/hexane to give the intermediate 12 in 87% yield.

### Example 2

### Synthesis of Intermediate 13

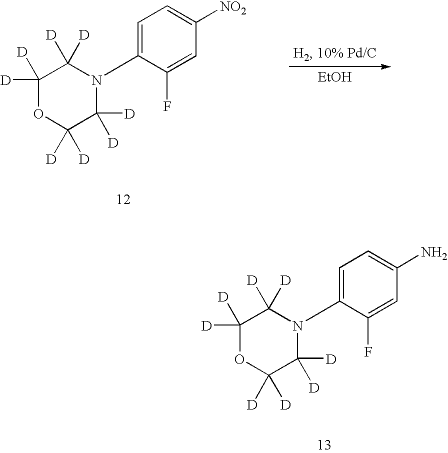

To a stirred suspension of 12 (50 g, 0.21 mol) in denatured EtOH (875 ml) under N₂ was added 10% Pd/C (50% wet, 17.5 g). The reaction vessel was purged with N₂ for 10 min, H₂ for 10 min and stirred overnight under an atmosphere of H₂. Hydrogenation was stopped and the vessel purged with N₂ for 15 min. The mixture was filtered through Celite, washed through with denatured EtOH (500 ml) and DCM (3×700 ml). The combined filtrates were concentrated in vacuo to give the desired aniline 13 (38.5 g, 90% yield) as a pink solid.

### Example 3

### Synthesis of (R)-d₂-epichlorohydrin 14B

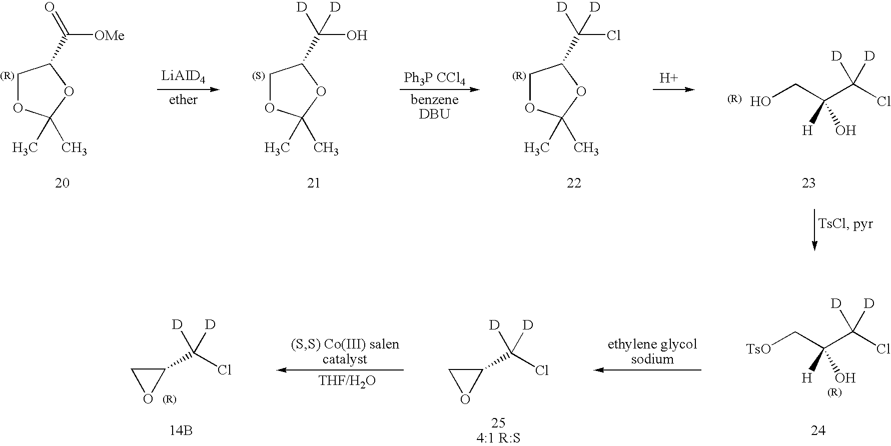

To an ice-cooled solution of methyl-α,β-isopropylidene-D-glycerate (20; 175 g, 1.09 mol, 1 equiv) in Et₂O (1000 ml) was added LiAlD₄ (34.43 g, 0.82 mol, 0.75 equiv) as a suspension in Et₂O (1000 ml) over 3 h. The reaction mixture was refluxed for 5 h. The mixture was allowed to cool to room temperature, diluted with Et₂O (1000 ml) and quenched with water (40 ml). The mixture was stirred for 15 min, filtered and the solid washed with Et₂O (1000 ml). The filtrate was concentrated in vacuo to give the corresponding alcohol 21 in high purity (121.3 g, 83% yield).

The alcohol (21; 60.65 g, 0.45 mol, 1 equiv) was dissolved in benzene (110 ml) together with PPh₃ (124 g, 0.47 mol, 1.05 equiv) and DBU (34 ml, 0.225 mol, 0.5 equiv). The mixture was added dropwise over 30 min to a refluxing solution of CCl₄ (110 ml) containing DBU (17 ml, 0.11 mol, 0.25 equiv). The reaction mixture was stirred at reflux overnight. The reaction mixture was allowed to cool to room temperature and concentrated in vacuo to give a crude mixture. The crude mixture was absorbed onto silica (60 g) and purified using column chromatography (silica: 1200 g) eluting with EtOAc:hexane 1:1 to give the desired chloride 22 in 54% yield.

The chloride 22 (37.6 g, 0.25 mol) was added to a solution of acetone (60 ml) and 1M HCl (150 ml). The mixture was heated to 55° C. for 30 min. The reaction mixture was allowed to cool to room temperature and the acetone removed in vacuo. The mixture was saturated with NaCl (43 g) and extracted with EtOAc (2×250 ml). The combined EtOAc layers were dried over MgSO₄ and concentrated to give the diol, (R)-d₂-chlorohydrin 23 (21.8 g, 79% yield).

To an ice-cooled solution of (R)-d₂-chlorohydrin 23 (21.0 g, 0.19 mol, 1 equiv) in pyridine (210 ml) was added portionwise toluenesulfonyl chloride (35.5 g, 0.19 mol, 1 equiv). After complete addition of the sulfonyl chloride the mixture was allowed to warm to room temperature and stirred for 1 h. Added to the mixture was Et₂O (300 ml) and the mixture washed with 1M HCl (3×500 ml) until the aqueous wash was acidic. The organic extract was washed with sat.aq. NaHCO₃ (300 ml), dried over MgSO₄ and concentrated to give the corresponding tosylate 24 (31.3 g, 63% yield).

Sodium metal (5 g, 37.48 mmol, 2 equiv) was added to ethylene glycol (40 ml), and the mixture stirred at 20° C. overnight to produce a solution of sodium ethylene glycolate in ethylene glycol. A solution of tosylate 24 (5 g, 18.74 mmol, 1 equiv) in ethylene glycol (5 ml) was then added, and the mixture stirred at 20° C. for 15 min. The product was removed from the mixture under reduced pressure and collected in a dry ice/IPA cold finger as a clear liquid to give enantiomerically enriched (R)-d₂-epichlorohydrin 25 (1.02 g, 58% yield). Chiral GC indicated an enantiomeric excess of 79.4%.

The (S,S)-Cobalt (II) catalyst (43.2 mg, 0.0716 mmol) was dissolved in toluene (8 PI). Acetic acid (8.6 μl, 0.143 mmol, 2 equiv) was added and the resulting mixture was stirred open to air at room temperature for 30 min, during which time the colour of the mixture changes from orange to dark brown. All volatile materials were removed in vacuo, affording the acetate complex of the Cobalt (III) catalyst as a brown residue. Added to the prepared catalyst was 80% enantiomerically enriched (R)-d₂-epichlorohydrin 25 (2.5 g) and THF (2.5 ml). The reaction flask was cooled to 0° C., and H₂O (0.05 ml) was added dropwise over 15 min. The reaction was allowed to warm to room temperature and stirred for 18 h. Added to the reaction mixture was a portion of MgSO₄ and the (R)-d₂-epichlorohydrin 26 was isolated by distillation at room temperature to give a 1:1 mix of epichlorohydrin and THF. Chiral GC analysis indicated an ee of 99.1%.

### Example 4

### Synthesis of Intermediates 15A and 15B

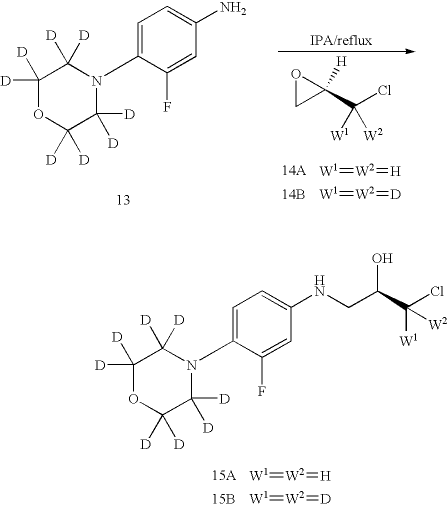

Aniline 13 (15 g, 74 mmol, 1.0 equiv.) was dissolved in isopropanol (75 ml) under N₂, and (R)-epichlorohydrin (14A; 7.0 g, 81.4 mmol, 1.1 equiv.) added. The mixture was stirred at reflux overnight. The solvent was removed in vacuo and the residue purified by column chromatography (750 g silica, CH₂Cl₂, then 1% MeOH, CH₂Cl₂) to give 15A as a pale brown oil (12.8 g, 58% yield).

Aniline (4.1 g, 20.1 mmol, 1.0 equiv.) was dissolved in isopropanol (20 ml) under N₂, and (R)-d₂-epichlorohydrin (14B; 2.0 g, 22.1 mmol, 1.1 equiv.) added. The mixture was stirred at reflux overnight. The solvent was removed in vacuo and the residue purified by column chromatography (300 g silica, CH₂Cl₂, then 1% MeOH, CH₂Cl₂) to give 15B as a pale brown oil (3.0 g, 50% yield). LC indicated a purity of 97%. Chiral LC indicated an enantiomeric excess of 95.7%.

### Example 5

### Synthesis of Intermediates 17A and 17B

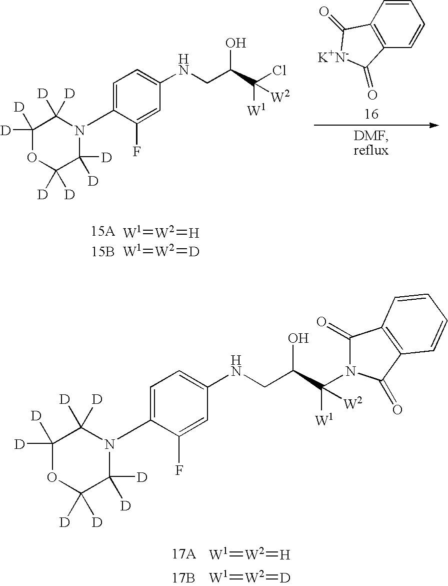

Intermediate 15A (12.8 g, 0.043 mol, 1 equiv), potassium phthalimide (16; 10.4 g, 0.056 mol, 1.3 equiv) and DMF (100 ml) was heated to 100° C. for 5 h. LC analysis indicated complete reaction. The reaction mixture was cooled to room temperature and poured into water (450 ml). The mixture was stirred for 2 h, filtered and the solid cake dried in a vacuum oven at 40° C. overnight to give 17B (9 g, 51% yield). LCMS indicated a purity of 95.0%.

Intermediate 15B (3.2 g, 10.7 mmol, 1 equiv), potassium phthalimide (16; 2.58 g, 13.9 mmol, 1.3 equiv) and DMF (23 ml) was heated to 100° C. for 5 h. The reaction mixture was cooled to room temperature and poured into water (100 ml). The mixture was stirred for 2 h, filtered and the solid cake dried in a vacuum oven at 40° C. overnight to give 17B (2.27 g, 52% yield). LC indicated a purity of 98.2%. Chiral LC indicated an enantiomeric excess of 98.6%.

### Example 6

### Synthesis of Intermediates 18A and 18B

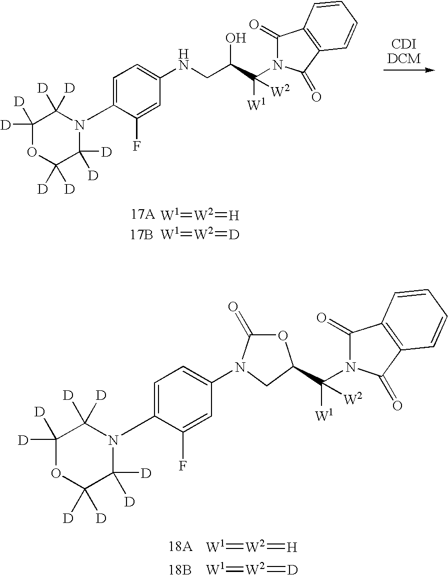

Intermediate 17A (9.0 g, 22 mmol, 1 equiv) was dissolved in DCM (100 ml), carbonyl diimidazole (5.0 g, 31 mmol, 1.4 equiv) was added at room temperature and the mixture was stirred overnight under nitrogen. LC analysis indicated the reaction was complete. Water (300 ml) was added to the mixture and the aqueous extracted with DCM (300 ml). The combined DCM layers were dried over MgSO₄ and concentrated in vacuo to give 18A. LCMS indicated a purity of 94.4%.

Intermediate 17B (2.08 g, 5.08 mmol, 1 equiv) was dissolved in DCM (25 ml), carbonyl diimidazole (1.15 g, 7.11 mmol, 1.4 equiv) was added at room temperature and the mixture was stirred overnight under nitrogen. LC analysis indicated the reaction was complete. Water (70 ml) was added to the mixture and the aqueous extracted with DCM (70 ml). The combined DCM layers were dried over MgSO₄ and concentrated in vacuo to give 18B (2.1 g, 95% yield). Chiral LC indicated an enantiomeric excess of 99.0%

### Example 7

### Synthesis of Intermediates 19A and 19B

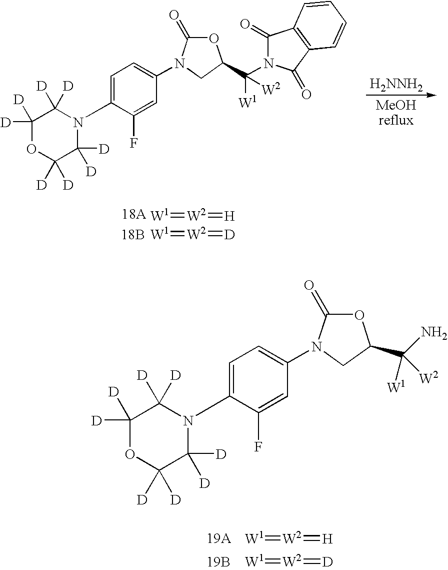

MeOH (100 ml) and hydrazine hydrate (6.1 ml, 0.125 mol, 5.5 equiv) were added to a flask containing 18A (9.9 g, 23 mmol, 1 equiv). The mixture was stirred at reflux temperature for 1 h. The reaction mixture was allowed to cool to room temperature, water (200 ml) was added, and the mixture was extracted with DCM (2×200 ml). The combined DCM extracts were washed with water (100 ml), dried over MgSO₄ and concentrated in vacuo to give 19A (6.0 g, 87% yield). LCMS indicated a purity of 96.8%.

MeOH (20 ml) and hydrazine hydrate (1.4 ml, 26.5 mmol, 5.5 equiv) were added to a flask containing 18B (2.1 g, 4.8 mmol, 1 equiv). The mixture was stirred at reflux temperature for 1 h. The reaction mixture was allowed to cool to room temperature, water (40 ml) was added, and the mixture was extracted with DCM (2×40 ml). The combined DCM extracts were washed with water (100 ml), dried over MgSO₄ and concentrated in vacuo to give 19B (1.26 g, 86% yield). Chiral LC indicated an enantiomeric excess of 99.3%.

### Example 8

### Synthesis of Intermediates 20A and 20B

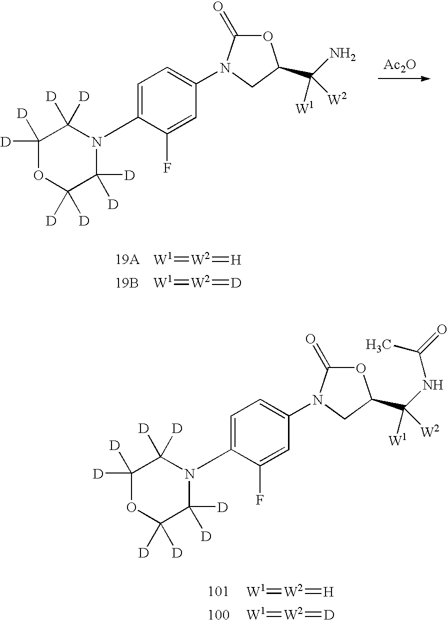

Intermediate 19A (6.0 g, 0.02 mol, 1 equiv) was stirred in toluene (90 ml) for 15 min. Acetic anhydride (5.4 ml, 0.057 mol, 2.9 equiv) was added dropwise at room temperature. The mixture was warmed to 35° C. with a water bath for 5 min to enhance the solubility of 19A. The reaction mixture was then stirred at room temperature for 1 h, was cooled to 0° C. and filtered to give compound 101 (5.3 g, 78% yield). LC indicated a purity of 99.1%. Chiral LC indicated an enantiomeric excess of 99.4%. ¹H-NMR (300 MHz, CDCl₃): δ 2.03 (s, 3H), 3.61-3.66 (m, 2H), 3.71-3.77 (m, 1H), 4.00 (apparent t, J 8.9, 1H), 4.71-4.79 (m, 11H), 6.50-6.54 (m, 1H), 6.88 (apparent t, J=8.9, 1H), 7.04 (dd, J₁=10.0, J₂=1.6, 1H), 7.41 (dd, J₁=14.6, J₂=2.7, 1H). HPLC (method: RP80A, 150 mm×4.6 mm column−gradient method 5-95% ACN+0.1% formic acid, with 5 min hold at 5% ACN prior to gradient, gradient over 10 mins, followed by 10 min hold at 95% ACN; T=30° C.; Wavelength: 258 nm): retention time: 11.45 min. Chiral HPLC (method: Chiralpak AD-H; 250×4.6 mm column; 5 μm particle size−hexane/IPA/DEA (80:20:01); T=40° C.; Wavelength: 258 nm): 11.80 min for desired enantiomer, 14.21 min for minor enantiomer; ee=99.8%. MS (M+H+): 346.5.
Intermediate 19B (1.25 g, 4.09 mmol, 1 equiv) was stirred in toluene (20 ml) for 15 min. Acetic anhydride (1.12 ml, 11.86 mol, 2.9 equiv) was added dropwise at room temperature. The mixture was warmed to 35° C. with a water bath for 5 min to enhance the solubility of 19B. The reaction mixture was then stirred at room temperature for 1 h, was cooled to 0° C. and filtered to give compound 100 (1.05 g, 74% yield). LC indicated a purity of 99.4%. Chiral LC indicated an enantiomeric excess of 98.9%. ¹H-NMR (300 MHz, CDCl₃): δ 2.00 (s, 3H), 3.74 (dd, J₁=9.1, J₂=6.8, 1H), 4.00 (t, J=9.1, 1H), 4.75 (dd, J₁=8.8, J₂=6.8, 1H), 6.52 (bs, 1H), 6.88 (t, J=8.8, 1H), 7.02-7.06 (m, 1H), 7.41 (dd, J₁=4.5, J₂=2.6, 1H). HPLC (method: RP80A, 150 mm×4.6 mm column−gradient method 5-95% ACN+0.1% formic acid, with 5 min hold at 5% ACN prior to gradient, gradient over 10 mins, followed by 10 min hold at 95% ACN; T=30° C.; Wavelength: 258 nm): retention time: 11.55 min. Chiral HPLC (method: Chiralpak AD-H; 250×4.6 mm column; 5 μm particle size−hexane/IPA/DEA (80:20:01); T=40° C.; Wavelength: 258 nm): 10.63 min for desired enantiomer, 13.18 min for minor enantiomer; ee=98.9%. MS (M+H+): 348.3.

### Example 9

### Synthesis of 2,2,6,6-d₄-morpholine (33) and Perdeuteromorpholine (10)

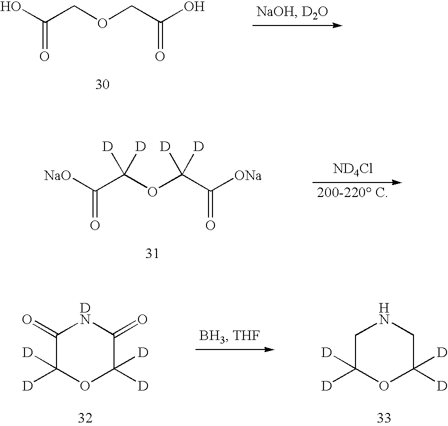

Diglycolic acid (30) is treated with sodium hydroxide in D₂O to produce the corresponding deuterated disodium compound 31. Compound 31 is then heated in the presence of perdeuteroammonium chloride to produce the d₄-dioxomorpholine 32, which is then reduced by boron trihydride in THF to produce the desired 2,2,6,6-d₄-morpholine (33). The tetradueteromorpholine 33 can be used in place of perdeuteromorpholine 10 in Example 1, above, to produce compounds of formulae I and Ia wherein each Z is deuterium and each Y is hydrogen, such as compounds 102 and 103.

### Example 10

### Antimicrobial Activity was Tested In Vivo Using the Murine Assay Procedure

Groups of female mice (six mice of 18-20 grams each) are injected intraperitoneally with Staphylococcus aureus bacteria which are thawed just prior to use and suspended in brain heart infusion with 4% brewers yeast (Staphylococcus aureus) or brain heart infusion (Streptococcus species). Antibiotic treatment at six dose levels per drug is administered one hour and five hours after infection by either oral intubation or subcutaneous routes. Survival was observed daily for six days. ED₅₀ values based on mortality ratios are calculated using probit analysis. The subject compounds are compared against a control (e.g., vancomycin).

### Example 11

The in vitro activity experiments are conducted by standard dilution methods known to those skilled in the art. Briefly, serial two-fold dilutions of antibiotic are prepared in a diluent, and a standard volume of mycobacterial growth medium is added to drug aliquot. The medium is inoculated with a standardized mycobacterial cell suspension, and then incubated under appropriate conditions. Following incubation, the Minimal Inhibitory Concentration (MIC) is determined by visual observation. The MIC is defined as the lowest drug concentration (in μg/ml) required to inhibit mycobacterial growth.

### Example 12

In vivo data is obtained from CD-1 mice infected intravenously with 1×10⁷ viable M. tuberculosis (Erdman strain). Twenty-four hours later drug treatment is initiated. All the drugs are given by oral gavage twice daily for four weeks. At the end of therapy, viable cell counts are determined from homogenates of spleens and lungs.

### Example 13

### Pharmacokinetics in Chimpanzees

The pharmacokinetics of compound 100 when administered to chimpanzees orally or intravenously in a 50:50 mixture with linezolid was studied. The solution for both oral and intravenous administration was prepared by combining linezolid (200 mg), compound 100 (200 mg), sodium citrate dihydrate (164 mg), anhydrous citric acid (85 mg), and dextrose monohydrate (5.024 g) in 900 ml of sterile water for injection at 65° C. with stirring. The mixture was cooled to 25° C. and the pH of the resulting solution adjusted to 4.8 with either 10% HCl or 10% NaOH as needed. The final volume of the solution was brought up to 111 with sterile water. The dosing solution is then filtered through a 0.22 μm filter prior to dosing.

Four chimpanzees (two male and two female) were used in the study. One male and one female were used for the intravenous study and one male and one female were used for the oral dosing study. All animals were fasted overnight prior to dosing. For all studies animals were sedated with ketamine (approx. 10 mg/ml) or telazol (approx. 5 mg/ml) prior to dosing. For each study animals were dosed with 300 mg of the combined drugs (150 mg each of linezolid and compound 100). Intravenous doses were administered (150 ml at 2 mg/ml combined drugs) by infusion over a 30 minute period. Oral doses were administered in a volume of 150 ml at 2 mg/ml combined drugs.

For the intravenous study, a 4.5 ml aliquot of blood was taken from each animal prior to the start of infusion, 15 minutes after the start of infusion, and immediately before the end of infusion. Additional samples were taken at 6, 15, 30, and 60 minutes and 1.5, 2, 4, 6, 8 and 24 hours following infusion. For the oral study, 4.5 ml aliquots of blood were taken from each animal prior to doing and then at 15, 20 and 60 minutes and 1.5, 2, 4, 6, 8 and 24 hours post dosing. All blood samples were collected into vacutainer tubes containing sodium heparin as an anticoagulant, sufficiently mixed and stored on wet ice. The samples were centrifuged within 1 hour of collection and the plasma collected and frozen at −70° C. until analysis. Urine was also collected from each animal over a 24 hour period following dosing.

Each sample was analyzed by LC-MS/MS for the presence of both linezolid and compound 100 as follows. Chimp plasma sample (100 μL) was mixed with 300 μL internal standard solution prior to LC-MS/MS analysis. The internal standard was 250 ng/mL haloperidol in acetonitrile/water (90/10, v/v). After protein precipitation, 10 μL supernatant was injected to a Zorbax SB-C8 (Rapid Resolution) column (2.1×30 mm, 3.5 μm). The initial mobile phase condition was 100% A (water with 0.1% formic acid) and 0% B (acetonitrile with 0.1% formic acid) with a flow rate at 0.5 mL/min. Mobile phase B was allowed to reach 90% within 2 minutes and held for 1 minute before ramping back 0% at 3.2 minutes. The overall run time was six minutes. The precursor/product ion pairs were set at m/z 338/296, m/z 348/306 and m/z 376/165 for detecting linezolid, Compound 100 and haloperidol, respectively.

Urine samples were similarly analyzed. Chimp urine samples (10 μL) were independently injected to a Zorbax SB-C8 (Rapid Resolution) column (2.1×30 mm, 3.5 μm). The initial mobile phase condition was 100% A (water with 0.1% formic acid) and 0% B (acetonitrile with 0.1% formic acid) with a flow rate at 0.4 mL/min. Mobile phase B was allowed to reach 25% within 42 minutes and then from 25% to 90% in two minutes before ramping back 0% in four minutes. The overall run time was 48 minutes. The mass spectrometer was set in positive ion mode and ions were scanned from m/z 100 to 1000. Once certain molecular ions of metabolites were identified, MS/MS experiments were carried out to produce product ions.

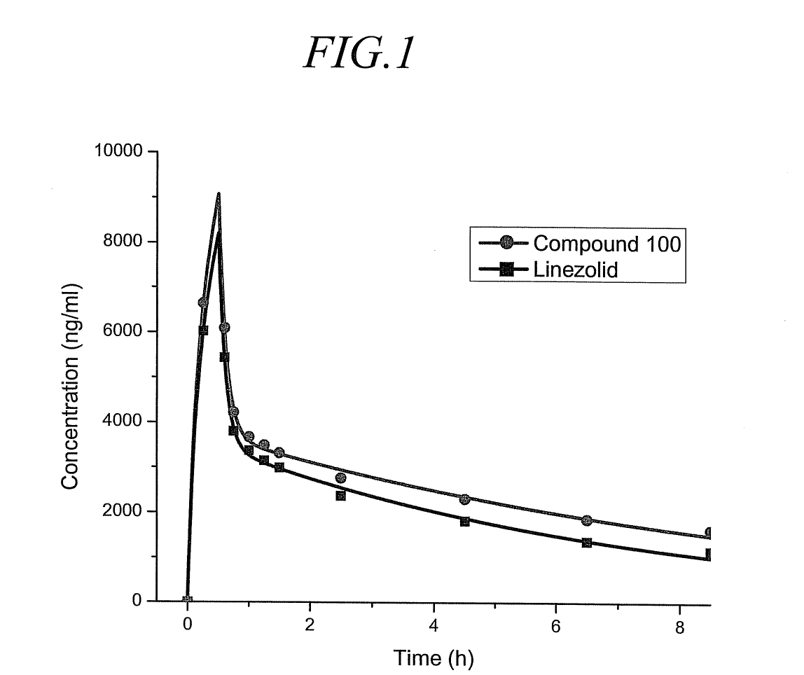

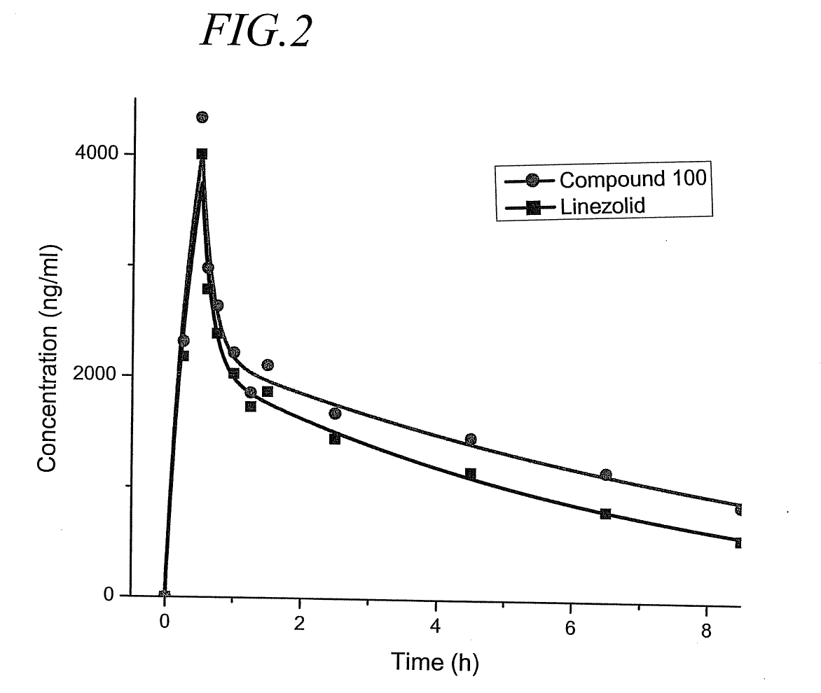

FIGS. 1 and 2 show the results of the intravenous dosing study. Both the female (FIG. 1) and male chimpanzee (FIG. 2) exhibited an increased half-life and AUC for compound 100 as compared to linezolid. The calculated half-lives for IV dosing are shown in Table 1.

TABLE 1
Half-lives of compound 100 and linezolid following intravenous dosing

| Drug | Half-life (Female) | Half-life (Male) |
|---|---|---|
| Linezolid | 4.5 h | 4.5 h |
| Compound 100 | 6.4 h | 6.2 h |

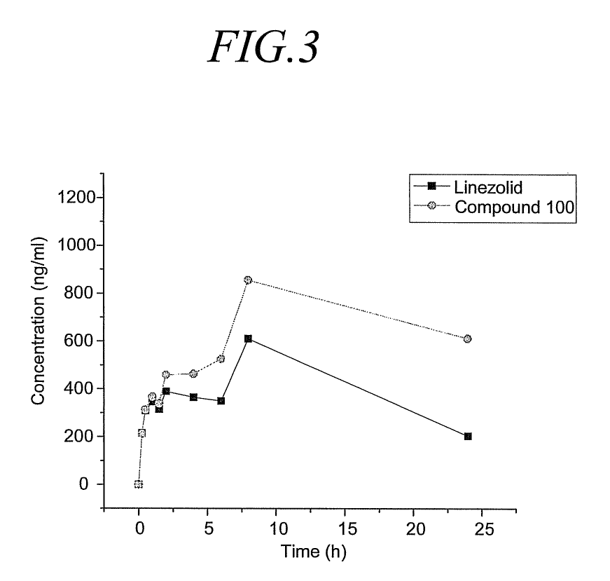

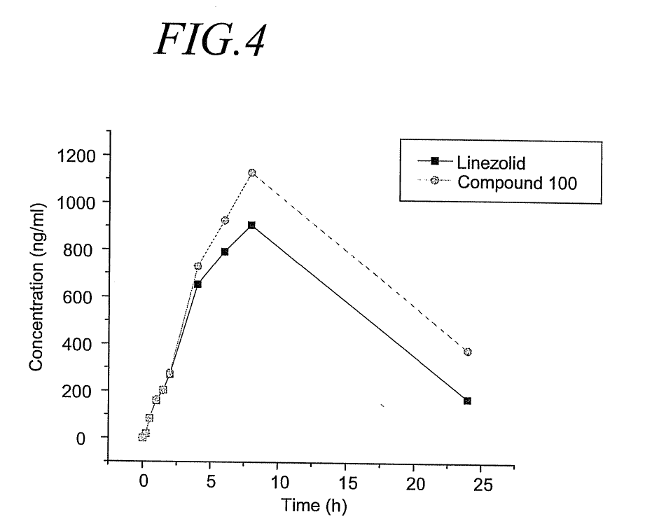

FIGS. 3 and 4 show the results of the oral dosing study. Both the female (FIG. 3) and male chimpanzee (FIG. 4) exhibited an increased half-life and AUC for compound 100 as compared to linezolid. The ratio of serum concentration of compound 100 to linezolid at 8 and 24 hours is shown in Table 2. The mean calculated AUC for each compound is set forth in Table 3.

TABLE 2
Ratio of serum concentration of compound 100 to linezolid following
oral dosing.

| | Ratio (compound | Ratio (compound |
|---|---|---|
| Time post-dosing | 100:linezolid) Female | 100:linezolid) Male |
| 8 h | 1.39 | 1.20 |
| 24 h | 2.99 | 2.20 |

TABLE 3
Mean AUC_{0-24 h} of compound 100 and linezolid
following oral dosing

| Compound | Mean AUC_{0-24 h} (ng * hr/ml) |
|---|---|
| Linezolid | 11300 |
| Compound 100 | 16400 |

The metabolic fate of compound 100 as compared to linezolid was analyzed by following excretion of each compound in the urine after intravenous or oral dosing. The results of this analysis are set forth in Table 4.

TABLE 4
Excretion of intact linezolid and compound 100 in urine.

| | |
|---|---|
| Intravenous | Oral |

| | Male | Female | Male | Female |
|---|---|---|---|---|
| Linezolid | 9290 | 17200 | 13300 | 12800 |
| Compound 100 | 18100 | 35000 | 23900 | 20000 |

The results shown in Table 4 demonstrate that approximately twice as much Compound 100 was excreted intact in the urine as linezolid, regardless of the route of administration or the sex of the subject. In addition, further analysis demonstrated that the amount of the M6 metabolite and its deuterated equivalent were essentially the same, while the amount of deuterated M4 metabolite was significantly lower than the linezolid M4 metabolite.

The chimpanzee studies indicate that compound 100 is more slowly metabolized than linezolid and that its metabolic fate is shifted away from the M4 metabolite to intact excretion as compared to linezolid.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. Various modifications and equivalents can be made without departing from the spirit and scope of the invention.

1. A compound of formula I or Ia: 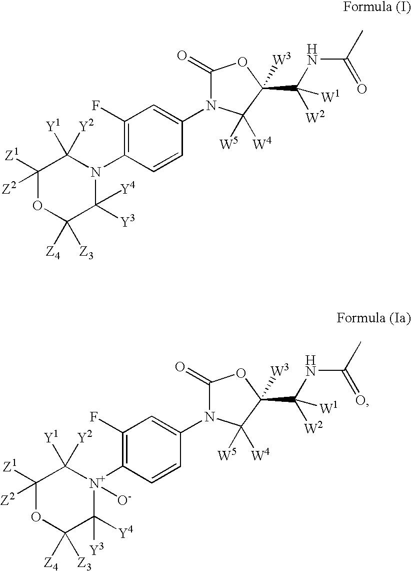
or a pharmaceutically acceptable salt thereof, wherein:
each W is independently hydrogen or deuterium;
each Y is independently hydrogen or deuterium;
each Z is independently hydrogen, deuterium, or fluorine; and
at least one W, Y or Z is deuterium.

2. The compound of claim 1, wherein:
at least 1 W is deuterium;
at least 2 Y moieties are deuterium; and
at least 2 Z moieties are deuterium or fluorine.

3. The compound of claim 1, wherein W¹ and W² are simultaneously deuterium.

4. The compound of claim 1, wherein W¹ and W² are simultaneously hydrogen.

5. The compound of claim 1, wherein Y¹, Y², Y³ and Y⁴ are simultaneously deuterium.

6. The compound of claim 1, wherein Y′, Y², Y³ and Y⁴ are simultaneously hydrogen.

7. The compound of claim 1, wherein each of Z¹, Z², Z³ and Z⁴ is independently selected from deuterium and fluorine.

8. The compound of claim 7, wherein Z¹, Z², Z³ and Z⁴ are simultaneously deuterium.

9. The compound of claim 1, wherein the configuration of the compound of Formula I or Ia is (S).

10. The compound of claim 1 selected from the group consisting of:

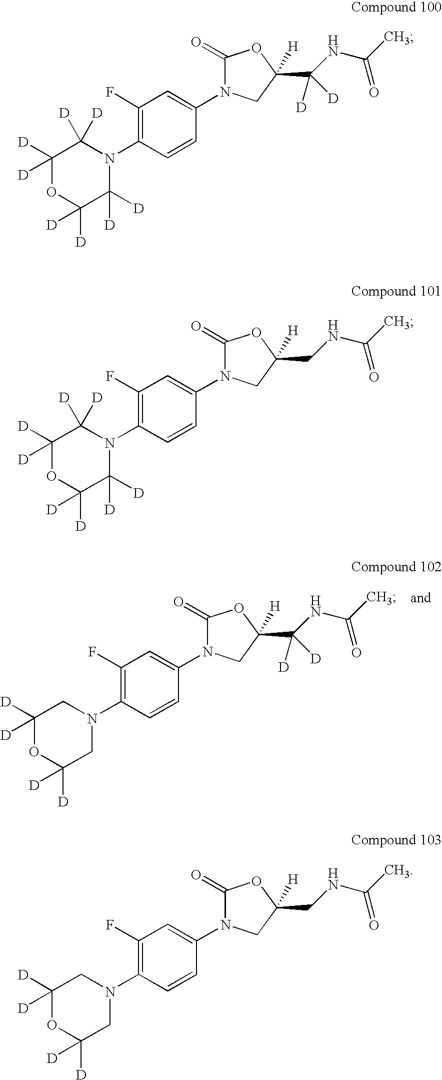

11. The compound of claim 1, wherein any atom not designated as deuterium is present at its natural isotopic abundance.

12. A pyrogen-free composition comprising a compound of claim 1 and an acceptable carrier.

13. The composition of claim 12 formulated for pharmaceutical administration, wherein the carrier is a pharmaceutically acceptable carrier.

14. The composition of claim 13, further comprising a second therapeutic agent selected from an anti-microbial agent and a cyclooxygenase inhibitor.

15. The composition of claim 14, wherein the second therapeutic agent is selected from gentamicin, tobramycin, aztreonam, cefazolin, ceftazidime, piperacillin, ciprofloxacin, ofloxacin, levofloxacin, celecoxib, and rofecoxib.

16. A method of treating a subject suffering from or susceptible to a bacterial infection or a fungal disorder comprising the step of administering to the subject in need thereof a composition of claim 11.

17. The method of claim 16, wherein the subject is suffering from or susceptible to an infection caused by a bacteria selected from Enterococcus faecium, Staphylococcus aureus, Streptococcus agalactiae, Streptococcus pneumoniae, Streptococcus pyrogenes, Enterococcus faecalis, Staphylococcus epidermidis, Staphyloccocus haemolyticus, and Pasteurella multocida.

18. The method of claim 16, wherein the subject is suffering from or susceptible to a disease or disorder selected from a Gram-positive bacterial infection, Vancomycin-resistant Enterococcus faecium infection; nosocomial pneumonia due to Staphylococcus aureus and Streptococcus pneumoniae; complicated skin and skin structure infections caused by Staphylococcus aureus, Streptococcus pyogenes, or Streptococcus agalactiae; uncomplicated skin and skin structure infections caused by Staphylococcus aureus or Streptococcus pyogenes; community-acquired pneumonia caused by Streptococcus pneumoniae or Staphylococcus aureus; and tuberculosis.

19. The method of claim 18, wherein the subject is suffering from or susceptible to a disease or disorder selected from a Gram-positive bacterial infection, Vancomycin-resistant Enterococcus faecium infection; nosocomial pneumonia due to Staphylococcus aureus and Streptococcus pneumoniae; complicated skin and skin structure infections caused by Staphylococcus aureus, Streptococcus pyogenes, or Streptococcus agalactiae; uncomplicated skin and skin structure infections caused by Staphylococcus aureus or Streptococcus pyogenes; and community-acquired pneumonia caused by Streptococcus pneumoniae or Staphylococcus aureus

20. The method of claim 16 comprising the additional step of administering to the subject in need thereof a second therapeutic agent selected from gentamicin, tobramycin, aztreonam, cefazolin, ceftazidime, piperacillin, ciprofloxacin, ofloxacin, levofloxacin, celecoxib, and rofecoxib.
